(19) United States
(12) Patent Application Publication
(10) Pub. No.: US 2016/0011177 A1
(43) Pub. Date: Jan. 14, 2016

(54) SCREENING METHOD FOR THERAPEUTIC AGENTS FOR CHARCOT-MARIE-TOOTH DISEASE AND SELF-DIFFERENTIATION MOTOR NEURONS USED THEREFOR

(71) Applicants: Samsung Life Public Welfare Foundation, Seoul (KR); Chong Kun Dang Pharmaceutical Corp, Seoul (KR)

(72) Inventors: Yuntae Kim, Gyeonggi-do (KR); Byung-Ok Choi, Seoul (KR); So-Youn Woo, Seoul (KR); Ji-Yon Kim, Seoul (KR); Sung Chul Jung, Seoul (KR); Young Bin Hong, Seoul (KR); Jin-Mo Park, Seoul (KR)

(73) Assignees: CHONG KUN DANG PHARMACEUTICAL CORP, Seoul (KR); SAMSUNG LIFE PUBLIC WELFARE FOUNDATION, Seoul (KR)

(21) Appl. No.: 14/873,125

(22) Filed: Oct. 1, 2015

Related U.S. Application Data
(63) Continuation-in-part of application No. PCT/KR2014/002794, filed on Apr. 1, 2014.

(30) Foreign Application Priority Data
Apr. 2, 2013 (KR) 10-2013-0035739
Apr. 1, 2014 (KR) 10-2014-0038467

(51) Int. Cl. G01N 33/50; C12N 5/0793

(52) U.S. Cl. CPC G01N 33/5058; C12N 5/0619; C12N 2501/603; C12N 2501/602; C12N 2501/604; C12N 2501/606; C12N 2501/60; C12N 2501/41; C12N 2501/13; C12N 2506/1307; G01N 2500/10; G01N 2333/4703; G01N 2440/10

(57) ABSTRACT

The present invention relates to a method for the screening of a therapeutic agent for Charcot-Marie-Tooth disease (CMT) using induced pluripotent stem cells and motor neurons differentiated therefrom. Particularly, the present inventors prepared induced pluripotent stem cells from the human fibroblasts originated from CMT patient. When the motor neurons differentiated from the said induced pluripotent stem cells are used for the screening of a therapeutic agent for Charcot-Marie-Tooth disease, the pharmaceutical effect of the therapeutic agent candidates can be easily evaluated during the screening. In addition, by the method to prepare the induced pluripotent stem cells, autologous motor neurons which are usable for the screening of a patient-specific therapeutic agent and the patient-specific treatment can be prepared.

### CROSS REFERENCE TO RELATED APPLICATIONS

This is a continuation-in-part of PCT Application No. PCT/KR2014/002794, filed on Apr. 1, 2014 which claims priority to Korean Application No. 10-2014-0038467, filed on Apr. 1, 2014 and Korean Application No. 10-2013-0035739, filed on Apr. 2, 2013. The prior applications are all incorporated herein by reference,

### BACKGROUND OF THE INVENTION

1. Field of the Invention

The present invention relates to a method for the preparation of induced pluripotent stem cell and a method for the screening of a therapeutic agent for Charcot-Marie-Tooth disease using the autologous cells differentiated from the same.

2. Description of the Related Art

Charcot-Marie-Tooth disease (CMT) or hereditary motor and sensory neuropathy is the defect or damage in motor neurons and sensory neurons resulted from specific gene mutation. Hereditary peripheral neuropathies can be classified into three groups which are hereditary motor and sensory neuropathies (HMSN), hereditary motor neuropathies (HMN), and hereditary sensory neuropathies (HSN). So, hereditary motor and sensory neuropathy is one of them. Since this disease was first identified in 1886 by Charcot, Marie, and Tooth, the disease was named after them, “Charcot-Marie-Tooth” disease, or has been simply called CMT after their first initials of their names. In the late 20^{th} century, Dyck, et al. called the CMT another name ‘hereditary motor and sensory neuropathy (HMSN)’ and thereafter the disease is now called both CMT or HMSN. Charcot-Marie-Tooth disease is divided into many groups according to the hereditary pattern, which are autosomal dominant inherited type I and type II, autosomal recessive inherited type IV, and X-chromosome linked inherited type CMTX. Type I members were named as 1A, 1B, 1C, and so on according to the gene mutation reporting order.

The incidence rate of Charcot-Marie-Tooth disease is 1/2500 people, which is rather high among rare hereditary diseases. Charcot-Marie-Tooth disease patients show such symptoms that their hand/foot muscles are getting weaker and weaker and their hands and feet are often deformed. The degree of the symptoms vary according to the type of gene mutation. Some patients display as light symptoms as almost close to the normal people and some patients show severe symptoms so much as they need help with walking or have to sit on wheel-chair.

The conventional treatment method for CMT is limited to rehabilitation, assistive technology devices, and pain control. However, the identification of CMT related genes made genetic counseling and family planning possible, based on which science-based clinical care is advancing. The actual treatment or help that can change the course of progress of hereditary motor and sensory neuropathies has not been established yet, but the possibility has been confirmed in the recent animal tests. Along with that, studies are still under-going on gene therapy, cell replacement therapy, axonal transport related therapy, mitochondrial function correction, immune system based therapy, and integrin therapy.

With the breath-taking advancement in the study of rare disease for the last few decades, there have been quantitative and qualitative changes in the treatment of the disease from the diagnosis to the treatment including practice guideline. In particular, the advancement of molecular biology made changes in diagnostic methods and accordingly targeted therapy represented by individualization or tailored therapy considering the different molecular biological origins of rare disease has been established. Also, the development of pharmacogenetics provided the vision that patients even with the same disease or on the same drugs can be treated differently considering their own genetic characteristics. So, we can call these days ‘the era of molecular genetics’. In particular, CMT is most exposed among rare diseases on a variety of treatment selection and prognosis including symptomatic treatment aiming at the relief of symptoms with pharmacotherapy and additional treatment and supportive therapy aiming at the relief and control of side effects and complications. CMT is resulted from gene malfunction, so the symptoms are continued and cannot be cured completely. The conventional treatment of CMT, therefore, is to relieve the symptoms and delay the progress in order to increase quality of life. Biological treatment has been continually attempted through genetic and molecular biological studies and some promising results have been reported. However, morbidity is rare due to the characteristics of the disease and interest to boost the study is also low, so a proper treatment method has not been established yet and doctors and researchers who can diagnose and design the treatment for such a rare disease are still short (Acta Paediatri, 2012).

In the transgenic mouse administered with the progesterone receptor antagonist ‘onapristone’, known as one of CMT treating drugs, the over-expression of Pmp22 mRNA was suppressed and the phenotype of hereditary motor and sensory neuropathies was improved without side effects, according to the previous report. Ascorbic acid, the essential material for myelination in peripheral nerves was functioning for remyelination and improved the phenotype of hereditary motor and sensory neuropathies in CMT1A transgenic mouse. It was also reported that neurotrophin-3 (NT-3) increased myelinated nerve fibers and as a result sensor related symptoms were improved. However, the above therapeutic materials are limited in CMT type 1 treatment. CMT is resulted from tens of different gene mutations. So, in order to treat such CMT in diversity, it is urgently requested to establish each gene defect tailored treatment method and a method to evaluate the newly established treatment method. The response to a drug is significantly different among CMT patients, so drug selection is limited since the symptoms are all different among CMT patients.

Stem cells obtained from skin tissue of a patient have the characteristics of gene mutation of the patient. Therefore, when the stem cells are differentiated into neurons, the neurons having all the disease characteristics of the patient can be obtained, which are expected to be useful for drug selection or patient-specific treatment.

Charcot-Marie-Tooth disease (CMT), the representative hereditary peripheral neuropathy, is a single gene disorder. The CMT disease model can be constructed by differentiation of the induced pluripotent stem cells originated from patient's skin cells. A novel therapeutic agent can be prepared by using such disease model that can re-produce the disease characteristics. The induced pluripotent stem cells originated from patients having spinal muscular atrophy, familial dysautonomia, or LEOPARD syndrome were used to reproduce the abnormality and symptoms of those patients in vitro. When the cultured cells were treated with those test drugs, the symptoms were improved (Ebert A D. et al, Nature, 2009, 457:277-280, Lee G. et al, Nature, 2009, 461:402-406, Cavajal-Vergara X. et al, Nature, 2010, 465:808-812, Hanna J. et al, Science, 2007, 318:1920-1923). Therefore, the induced pluripotent stem cells and the autologous cells differentiated from the same can be used for the approach to develop a patient specific novel drug for those who are suffering from those diseases that do not have a proper cure, suggesting that they can be helpful for those patients who have incurable rare disease.

### SUMMARY OF THE INVENTION

It is an object of the present invention to provide a method for preparing motor neurons from the somatic cells originated from Charcot-Marie-Tooth disease (CMT) patient.

It is another object of the present invention to provide a screening method for CMT treating agent candidates.

It is also an object of the present invention to provide CMT patient autologous motor neurons differentiated from the induced pluripotent stem cells prepared by the method of the invention.

It is further an object of the present invention to provide a screening method for a patient specific CMT type dependent therapeutic agent using the CMT patient autologous motor neurons prepared by the method of the invention.

To achieve the above objects, the present invention provides a method for preparing motor neurons from the somatic cells originated from Charcot-Marie-Tooth disease (CMT) patient.

The present invention also provides a screening method for CMT treating agent candidates.

The present invention further provides CMT patient autologous motor neurons differentiated from the induced pluripotent stem cells prepared by the method of the invention.

In addition, the present invention provides a screening method for a patient specific CMT type dependent therapeutic agent using the CMT patient autologous motor neurons prepared by the method of the invention.

### ADVANTAGEOUS EFFECT

The present invention provides a method for preparing induced pluripotent stem cells from the human fibroblasts originated from Charcot-Marie-Tooth disease (CMT), a screening method for CMT treating agent candidates by using the motor neurons differentiated from the said induced pluripotent stem cells that can be efficient in confirming the pharmaceutical effect of those candidates, and CMT patient autologous motor neurons prepared by the method for preparing induced pluripotent stem cells. The autologous motor neurons can be efficiently used for the screening of a patient specific drug and for the patient specific treatment.

In the course of study to establish a patient specific treatment method for Charcot-Marie-Tooth disease (CMT) patients, the present inventors first prepared induced pluripotent stem cells from the human fibroblasts originated from CMT patient. Then, the inventors further confirmed that a screening method for CMT treating agent candidates using the motor neurons differentiated from the said induced pluripotent stem cells could be useful for the confirmation of pharmaceutical effect of the candidates and further constructed autologous motor neurons by the method of the invention that could be used for the screening of a patient specific drug and accordingly for the patient specific treatment, leading to the completion of this invention.

### BRIEF DESCRIPTION OF THE DRAWINGS

The application of the preferred embodiments of the present invention is best understood with reference to the accompanying drawings, wherein:

FIG. 1 is a set of digital images illustrating the shape of human fibroblasts used in this invention.

FIG. 2 is a diagram illustrating the method of differentiation of motor neurons from CMT originated induced pluripotent stem cells (iPSCs).

FIG. 3 is a set of graphs illustrating the gene mutation of HSP27, the CMT causing gene, in CMT originated induced pluripotent stem cells (CMT-2F-iPSC).

FIG. 4 is a set of digital images illustrating the shape of CMT 2F-iPSC colony. This photo has been taken 20 days from the culture began and the cells were densely populated in the induced pluripotent stem cell colony.

FIG. 5 is a set of digital images illustrating the expression of CMT 2F-iPSC endogenous pluripotent gene.

FIG. 6 is a set of digital images illustrating the expression of CMT 2F-iPSC stemness marker protein.

FIG. 7 is a set of digital images illustrating the in vitro differentiation potency of embryoid body (EB) induced from CMT 2F-iPSC, wherein the expressions of the ectoderm marker Nestin, the mesoderm marker smooth muscle actin (SMA), and the endoderm marker α-fetoprotein (AFP) were confirmed.

FIGS. 8A-8D are a set of digital images illustrating the differentiation potency confirmed by the observation of in vivo CMT 2F-iPSC teratoma formation.

FIGS. 9A-9C. FIGS. 9a and 9b are a set of diagrams illustrating the expression of CMT 2F-MN marker protein differentiated from CMT 2F patient and the formation of neuromuscular junction;

FIG. 9A is a set of digital images illustrating the expressions of HB9, ISL1, SMI32, Tuj1, MAP2 Synapsin, and ChAT, the CMT 2F-MN marker proteins;

FIG. 9B is a set of bar graphs illustrating the ratio of SMI32 and MPA2 positive proteins in CMT 2F-MN; and

FIG. 9C is a graph illustrating the length of axon of CMT 2F-MN.

FIG. 10 is a set of digital images illustrating the formation of neuromuscular junction of CMT 2F-MN.

FIGS. 11A-11C illustrate the expression of acetylated α-tubulin as the CMT index for the investigation of axonal transport efficiency over the treatment of tubastatin A in CMT 2F-MN;

FIG. 11A is a set of digital images illustrating the acetylation of α-tubulin in CMT 2F-MN;

FIG. 11B is a digital image illustrating the result of Western blotting performed to confirm the acetylation of α-tubulin in CMT 2F-MN over the treatment of tubastatin A; and

FIG. 11C is a bar graph illustrating the quantification of α-tubulin acetylation in CMT 2F-MN over the treatment of tubastatin A.

FIGS. 12A-12D illustrate the moving mitochondria as the CMT index for the investigation of axonal transport efficiency over the treatment of tubastatin A in CMT 2F-MN;

FIGS. 12A-12B are digital images illustrating the axonal mitochondria in motor neurons observed through mito-RED2 introduced in CMT 2F-MN;

FIG. 12C is a bar graph illustrating the comparison of the moving speed of mitochondria in CMT 2F-MN over the treatment of tubastatin A; and

FIG. 12D is a bar graph illustrating the mitochondria migration in CMT 2F-MN over the treatment of tubastatin A, which is presented as %.

FIG. 13 is a diagram illustrating the microfluidic culture for the investigation of axonal transport efficiency in motor neurons of CMT 2F-MN.

### SEQUENCE LISTING

The Sequence Listing is submitted as an ASCII text file [7037-95837-01_Sequence_Listing.txt, Sep. 30, 2015, 3.41 KB], which is incorporated by reference herein.

### DESCRIPTION OF THE PREFERRED EMBODIMENTS

Hereinafter, the present invention is described in detail.

The present invention provides a method for preparing motor neurons from the somatic cells originated from Charcot-Marie-Tooth disease (CMT) patient which comprises the following steps:

1) obtaining human somatic cells from Charcot-Marie-Tooth disease (CMT) patient;

2) transfecting the human somatic cells originated from CMT patient of step 1) with a vector introduced with OCT4, SOX2, KLF4, and c-MYC transgenes, followed by culture to induce induced pluripotent stem cells (iPSCs); and

3) inducing motor neurons by culturing the induced pluripotent stem cells prepared in step 2) in the presence of retinoic acid and sonic hedgehog.

In step 1), the Charcot-Marie-Tooth disease (CMT) can be CMT type I, CMT type II, CMT type IV, or CMTX, and is preferably CMT 2F herein. CMT 2F is characterized by the mutation wherein the 404^{th} and the 545^{th} cytosines of heat-shock protein (HSP) 27 are substituted with thymine. The mutant protein herein is characterized by the substitution of the 135^{th} amino acid ‘serine’ of the wild type HSP27 with phenylalanine or the substitution of the 182^{nd} amino acid ‘proline’ with leucine.

In step 1), the human somatic cells are preferably fibroblasts, but not always limited thereto.

The vector in step 2) can be a viral vector using sendai virus, retrovirus, and lentivirus or a non-viral vector, and particularly sendai virus is preferably used herein.

The medium used for the culture of human somatic cells in order to obtain the induced pluripotent stem cells after the transfection can be any conventional medium for culture. For example, Eagle's MEM (Eagle's minimum essential medium, Eagle, H. Science 130:432 (1959)), α-MEM (Stanner, C. P. et al., Nat. New Biol. 230:52 (1971)), Iscove's MEM (Iscove, N. et al., J. Exp. Med. 147:923 (1978)), 199 medium (Morgan et al., Proc. Soc. Exp. Bio. Med., 73:1 (1950)), CMRL 1066, RPMI 1640 (Moore et al., J. Amer. Med. Assoc. 199:519 (1967)), F12 (Ham, Proc. Natl. Acad. Sci. USA 53:288 (1965)), F10 (Ham, R. G. Exp. Cell Res. 29:515 (1963)), DMEM (Dulbecco's modification of Eagle's medium, Dulbecco, R. et al., Virology 8:396 (1959)), DMEM/F12 mixture (Barnes, D. et al., Anal. Biochem. 102:255 (1980)), Way-mouth's MB752/1 (Waymouth, C. J. Natl. Cancer Inst. 22:1003 (1959)), McCoy's 5A (McCoy, T. A., et al., Proc. Soc. Exp. Biol. Med. 100:115 (1959)), and MCDB series (Ham, R. G. et al., In Vitro 14:11 (1978)), but not always limited thereto.

The induced pluripotent stem cells (iPSCs) in this invention are the cells that have pluripotency obtained from the artificial dedifferentiation of already differentiated cells, which are also called ‘dedifferentiated stem cells’ or ‘induced pluripotent stem cells’. The said induced pluripotent stem cells have almost the same characteristics as those of embryonic stem cells. Particularly, cell shape is similar and the expression patterns of genes and proteins are alike. The said iPSCs having pluripotency are also appropriate to confirm the pluripotency marker protein expression in vitro and display the teratoma formation in vivo. In particular, by introducing the iPSCs into the mouse blastocyst, chimera mouse can be generated and germline transmission can be possible. The iPSCs of the invention include all the human, monkey, pig, horse, cow, sheep, dog, cat, mouse, and rabbit originated iPSCs, but are preferably human originated iPSCs herein and most preferably CMT patient originated iPSCs.

The transgene in this invention indicates a gene or a genetic material that is transferred from an organism to another organism via natural migration or genetic engineering technique. Particularly, the DNA segment containing gene sequence that is separated from an organism and then introduced into another organism is an example. The gene sequence used for the transgene is introduced into a vector, which is exemplified by OCT4, SOX2, KLF4, and c-MYC. This transgene is required to dedifferentiate the already differentiated cells into induced pluripotent stem cells. The term ‘dedifferentiation’ in this invention indicates the epigenetic retrogression process that can reverse the already differentiated cells back to non-differentiated status so as to induce the cells to be differentiated another tissue, which is also called reprogramming process. This process is based on the reversibility of the epigenetic changes of genome. According to the purpose of the present invention, the said dedifferentiation includes all the process that can reverse the differentiated cells displaying 0%˜100% differentiation potency back to non-differentiated status. For example, the process that can reverse the fully differentiated cells that shows 0% differentiation potency back to the differentiated cells but still having differentiation potency of 1% can be included.

After step 3), the step of differentiating the induced pluripotent stem cells prepared above into motor neurons comprising the following substeps (3-1) and (3-2) can be preferably included, but not always limited thereto:

(3-1) culturing the induced pluripotent stem cells prepared above to obtain embryoid body (EB) and then differentiating the obtained EB into neurosphere; and

(3-2) differentiating the neurosphere prepared above into motor neurons.

The neurotrophin of step 4) is preferably selected from the group consisting of nerve growth factor (NGF), brain-derived neurotrophic factor (BDNF), neurotrophin-3 (NT-3), and glial cell-derived neurotrophic factor (GDNF), but not always limited thereto.

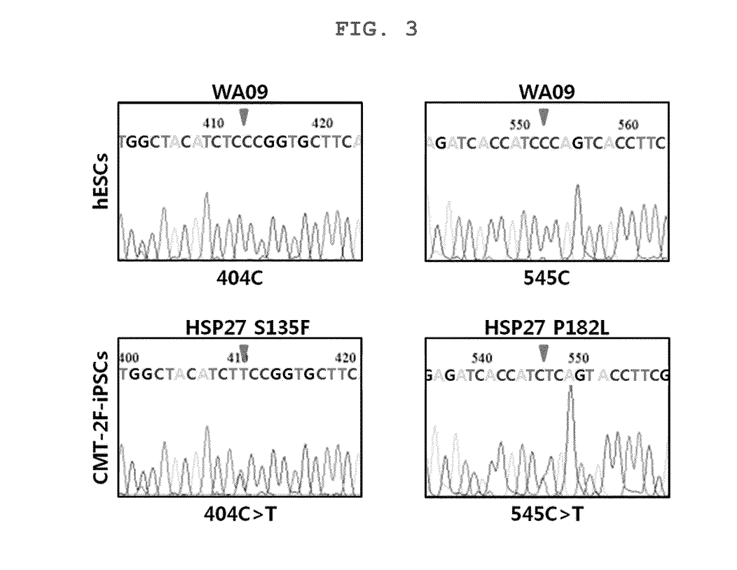

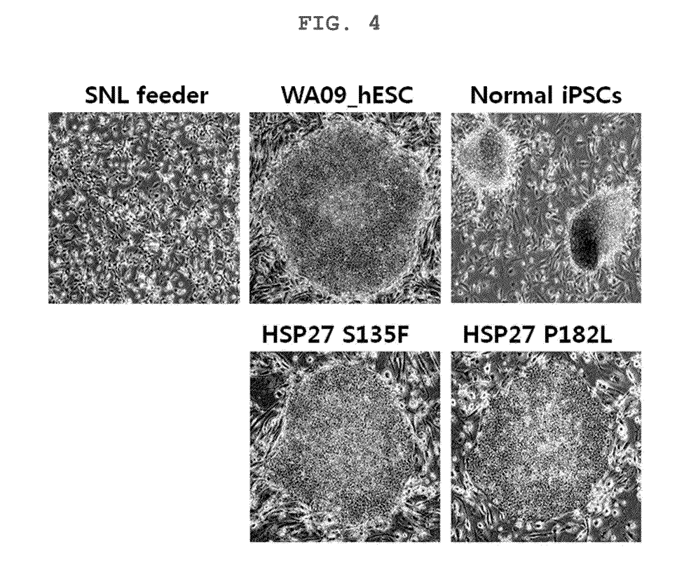

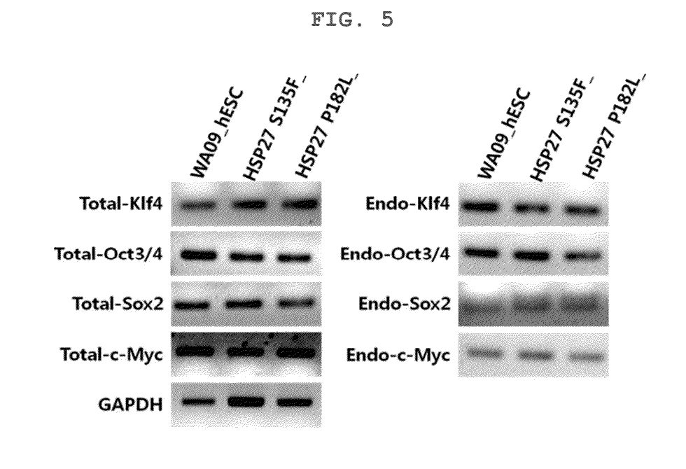

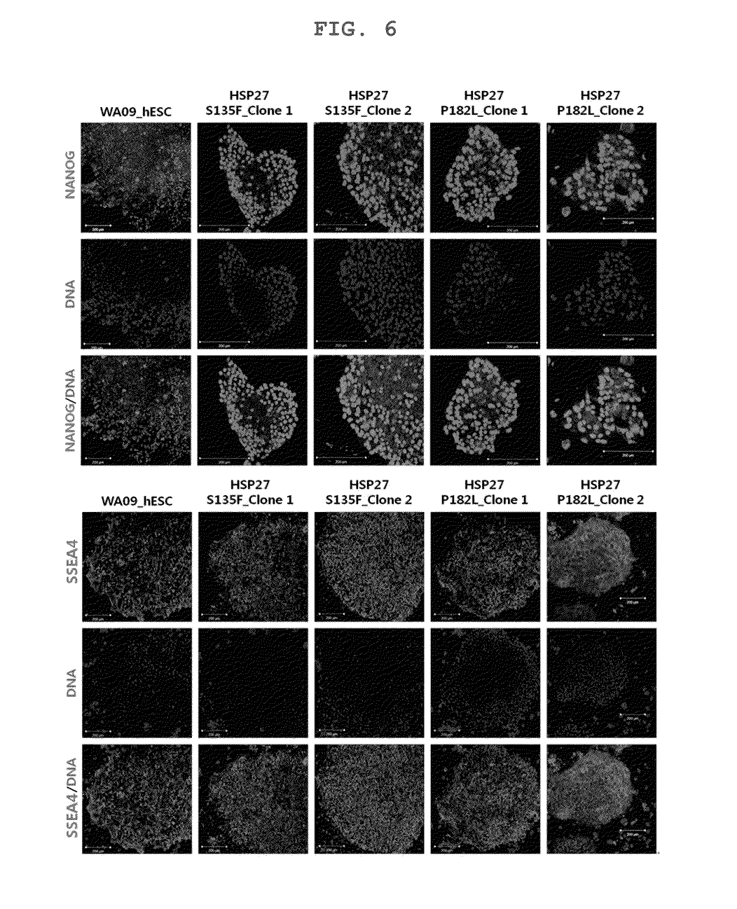

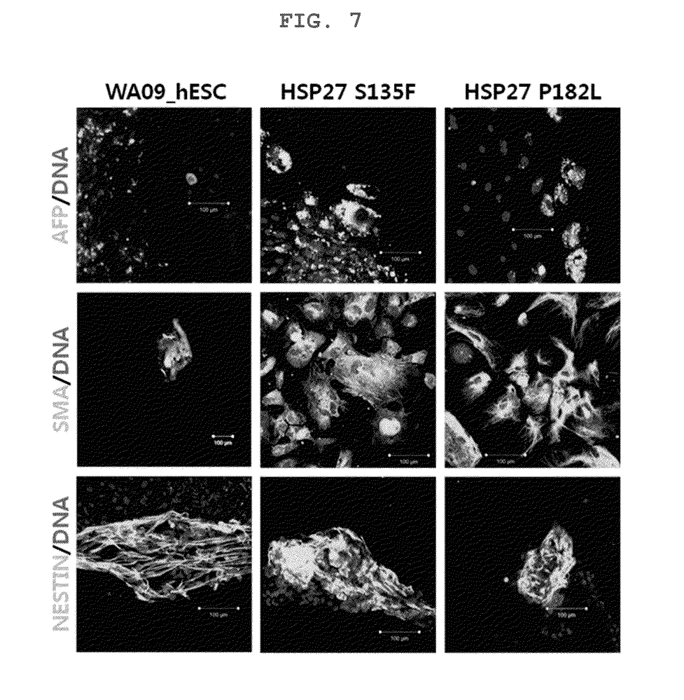

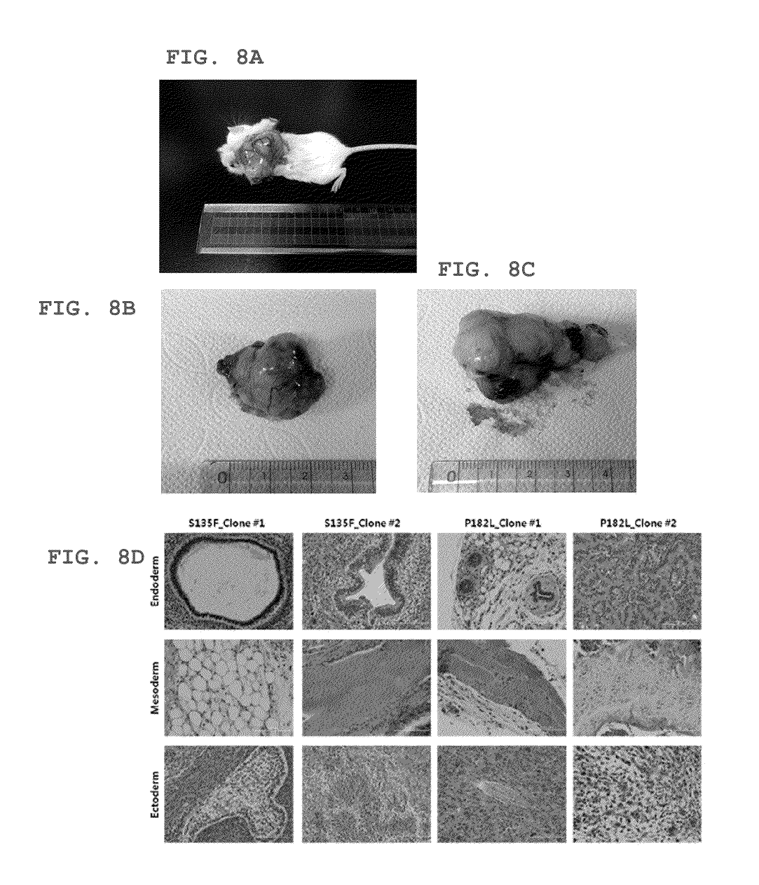

In a preferred embodiment of the present invention, the inventors prepared induced pluripotent stem cells (iPSCs) and embryoid body by using 4 kinds of transcription factors (Klf4, Oct3/4, Sox2, and c-Myc) from the fibroblasts obtained from skin biopsy of CMT 2F patient containing S135F or P182L mutation in HSP27 gene (see FIG. 4). The said iPSCs retain S135F or P182L mutation that has confirmed in CMT 2F patient (see FIG. 3) and are also able to express pluripotent marker gene and protein, suggesting that the prepared iPSCs and EB can be used as the CMT disease pluripotent stem cell model (see FIGS. 5 and 6). The EB differentiated from originated from CMT 2F patient derived iPSCs (CMT 2F-iPSC) was also confirmed to be differentiated into endoderm, mesoderm, and ectoderm in vitro (see FIG. 7) and to form teratoma in vivo (see FIG. 8).

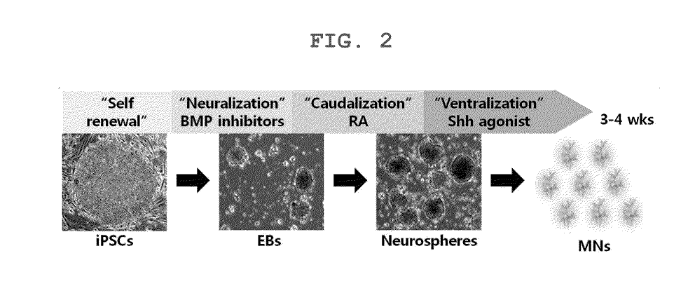

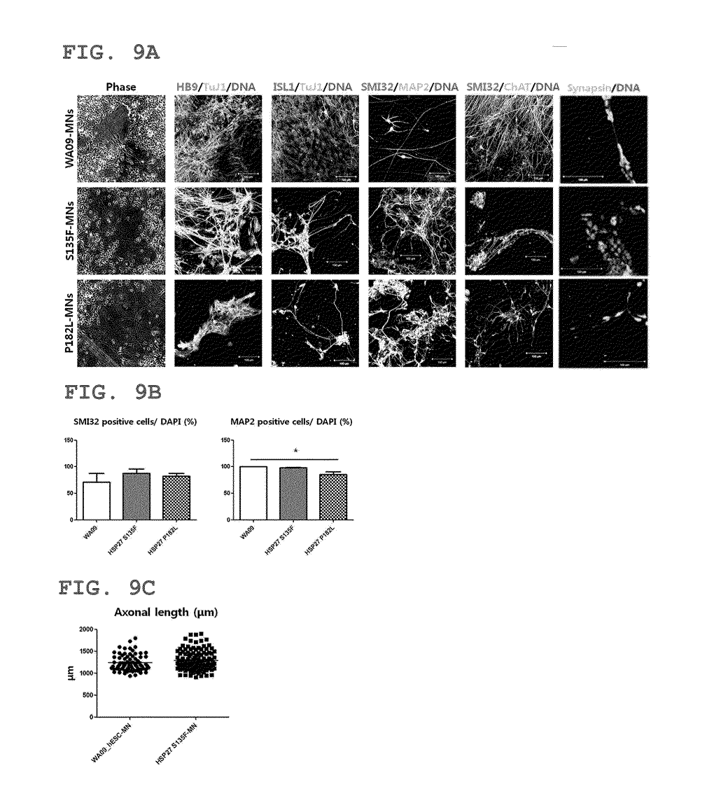

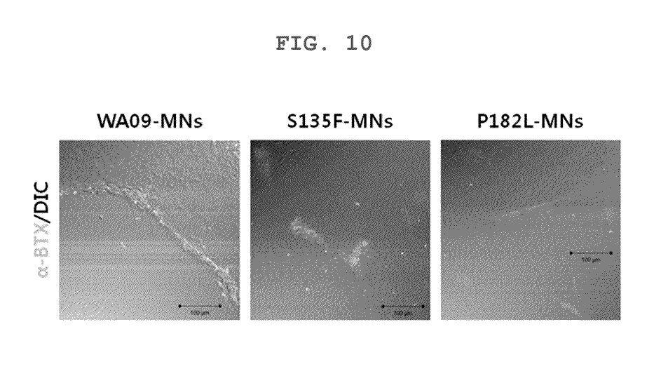

To use CMT 2F-iPSCs as the peripheral neuropathy model, the present inventors induced the differentiation of CMT 2F-iPSCs into motor neurons based on the informed method (Amoroso M W, et al, J Neurosci 2013; 33: 574-586) (see FIG. 2), and then investigated the differentiation efficiency by confirming the expression of motor neuron marker protein and the formation of neuromuscular junction (see FIGS. 9 and 10).

The CMT originated iPSCs model of the present invention not only contains the same mutation as the one found in CMT patient but also has pluripotency and can be efficiently differentiated into motor neurons through neurosphere, so that the method for preparing the said iPSCs model can be efficiently used for the study of CMT.

The present invention also provides a screening method for a composition for the prevention and treatment of Charcot-Marie-Tooth disease comprising the following steps:

1) treating the motor neurons prepared by the method of the invention with CMT treatment material candidates in vitro;

2) measuring the CMT index in the cells treated with the treatment material candidates in step 1); and

3) selecting the candidate that displays the increase or decrease of the CMT index obtained in step 2) by comparing with the control.

The present invention also provides a screening method for a patient specific CMT type dependent therapeutic agent.

The cells differentiated from the induced pluripotent stem cells prepared from CMT patient cells can be constructed by the above step 1)˜step 2) and step (3-1)˜(3-2).

The motor neurons differentiated from the CMT originated iPSCs of the invention can be used for the screening of CMT drug candidates. The said drug candidates include the histon deacetylase 6 (HDAC6) inhibitors Trichostatin, Tubacin, and tubastatin A, but not always limited thereto.

To measure cytotoxicity of the drug candidates, those candidates were treated to the normal control and CMT originated neurons at different concentrations and then the concentration that did not do harm on cell survival was determined. MTT (3-(4,5-dimethylthia-zol-2-yl)-2,5-diphenyltetrazolium bromide) test was performed to evaluate the cell survival rate.

After the CMT drug candidates were treated to the cells prepared above, CMT index was measured to investigate whether or not those drug candidates had usability as a drug. The said CMT index is preferably the axonal transport index, and particularly one or more indexes selected from the group consisting of acetylated α-tubulin, moving mitochondria, and action potential amplitude which is the electrophysiological index, and more preferably either or both acetylated α-tubulin or/and moving mitochondria, but not always limited thereto.

The present inventors confirmed that the concentration of acetylated α-tubulin was increased in the cells treated with the CMT drug candidates, suggesting that the selected candidates were efficient in treating CMT. At this time, when the level of acetylated α-tubulin was increased at least 20% higher than in the cells not-treated with the candidates, and preferably at least 30% higher, and more preferably at least 35% higher, it was judged that the candidate was efficient in treating CMT.

When moving mitochondria and action potential amplitude in the cells treated with the CMT drug candidate were recovered to the level of normal control neurons, the candidate was judged to be efficient in treating CMT.

At this time, the quantification of the protein expression can be performed by the various methods known to those in the art. For example, ELISA, Western blotting, or immunocytochemistry (ICC) can be used. The measurement of gene expression can be performed by RT-PCR (Sambrook et al., Molecular Cloning. A Laboratory Manual, 3rd ed. Cold Spring Harbor Press (2001)), northern blotting (Peter B. Kaufman et al., Molecular and Cellular Methods in Biology and Medicine, 102-108, CRC press), and hybridization using cDNA microarray (Sambrook et al., Molecular Cloning. A Laboratory Manual, 3rd ed. Cold Spring Harbor Press (2001)).

The Charcot-Mari-Tooth disease in step 1) can be CMT type I, CMT type II, CMT type IV, or CMTX, and is preferably CMT 2F. CMT 2F is characterized by the mutation wherein the 404^{th} and the 545^{th} cytosines of heat-shock protein (HSP) 27 are substituted with thymine. The mutant protein herein is characterized by the substitution of the 135^{th} amino acid ‘serine’ of the wild type HSP27 with phenylalanine or the substitution of the 182^{nd} amino acid ‘proline’ with leucine.

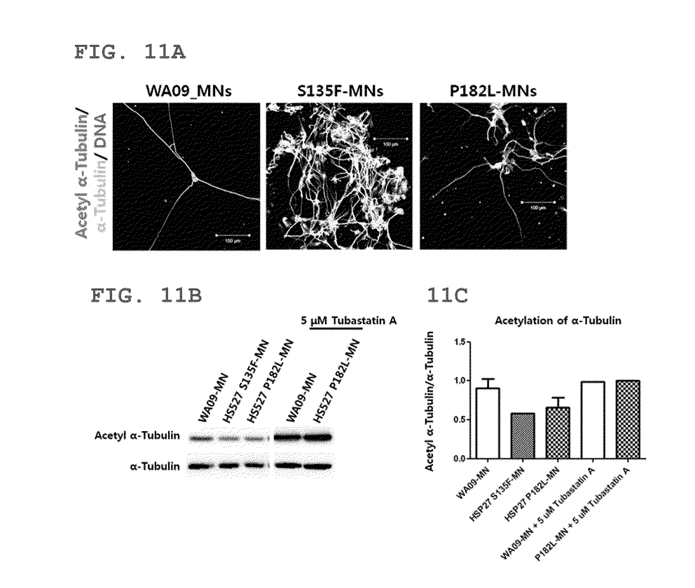

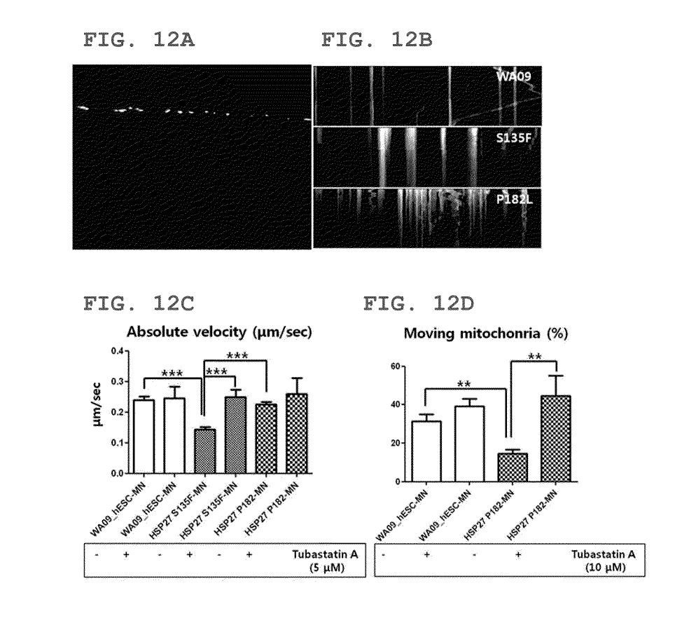

In another preferred embodiment of the present invention, the inventors used the neurons differentiated from CMT patient originated iPSCs as the CMT drug efficiency evaluation model in order to confirm the functions of microtubulin track involved in the axonal transport system defect, which is the major symptom of CMT 2F. To do so, the inventors investigated the efficiency of axonal transport in motor neurons of CMT 2F-MN by measuring the level of α-tubulin acetylation and moving mitochondria. As a result, in CMT 2F-MN, the level of α-tubulin acetylation was decreased, compared with in the normal control WA09_MN (see FIG. 11). Moving mitochondria was also reduced in CMT 2F-MN, compared with in the normal control (see FIG. 12). However, when the histon deacetylase 6 (HDAC6) inhibitor ‘tubastatin A’ was treated to CMT 2F-MN, the levels of α-tubulin acetylation and moving mitochondria were significantly increased, which were both recovered to the normal level of the normal control (see FIGS. 11b, 11c, 12b, and 12c).

The CMT patient originated iPSCs of the present invention contain the same mutation as the one that is a cause of CMT and at the same time can be differentiated into autologous motor neurons through neurosphere, and also facilitate the confirmation of decrease or increase of CMT index shown after the drug treatment without directly administering CMT drug candidates to patients, so that they enable the patient specific drug selection with displaying excellent effect and at the same time facilitate the selection of a drug that has least cytotoxicity.

Practical and presently preferred embodiments of the present invention are illustrative as shown in the following Examples.

However, it will be appreciated that those skilled in the art, on consideration of this disclosure, may make modifications and improvements within the spirit and scope of the present invention.

### EXAMPLE 1

### Separation of CMT Patient Originated Cells by Skin Biopsy

Skin biopsy is a safe low-invasive economical method for pathologic diagnosis of skin lesion. Under the approval of institutional review board, the inventors had an access to CMT 2F patients displaying the mutation of S135F or P182L in HSP27 gene and normal volunteers (Ewha Womans University Mokdong Hospital, Korea). To perform skin biopsy, normal volunteers and CMT patients were given local anesthesia and skin biopsy was performed by using a punch having a round blade in the diameter of 4 mm. The skin tissues obtained by skin biopsy were loaded in DMEM supplemented with 10 mg/ml collagenase type IV (Invitrogen, USA), 50 U/ml dispase (Roche), and 0.05% trypsin/EDTA, followed by reaction at 37° C. for 40 minutes. The obtained cell suspension was filtered by nylon cell strainer that can pass particles up to 70 μm in the size. The obtained fibroblasts were cultured in DMEM supplemented with 20% FBS and 100 μg/ml penicillin/streptomycin. Each sample was classified as shown in Table 1.

TABLE 1
Normal control group and CMT 2F patient group samples

| | | | HSP27 mutation | |
|---|---|---|---|---|
| | | | Nucleic | |
| | Classifi- | | acid | Protein |
| ID Number | cation | Cell line | mutation | mutation |
| WA09_hESC | Normal | WA09 human | — | — |
| | control | embryonic stem | | |
| | | cell line | | |
| Normal | Normal | Normal control | — | — |
| iPSC | control | group | | |
| | | originated | | |
| | | cells | | |
| HSP27 | CMT 2F | CMT 2F patient | 405 C > t | S135F |
| S135F | patient | originated | | |
| | group | cells | | |
| HSP27 | CMT 2F | CMT 2F patient | 545 C > T | P182L |
| P182L | patient | originated | | |
| | group | cells | | |

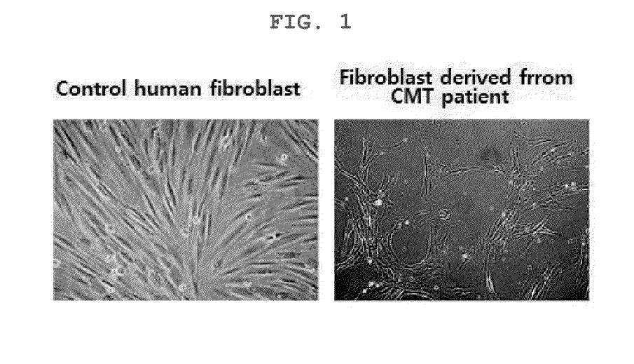

As a result, as shown in FIG. 1, the fibroblasts separated from the normal group and CMT patients were confirmed to be same in their morphology (FIG. 1).

### EXAMPLE 2

### Preparation of CMT Patient Originated Induced Pluripotent Stem Cells (iPSC) and Embryoid Body

<2-1> Inducement of the Development of iPSCs Originated from CMT Patient

To prepare iPSCs for the differentiation of neurons from the fibroblasts obtained from CMT patient by skin biopsy in Example 1, fibroblasts of normal control group and CMT patients were transfected with sendai virus system (Cell Biolabs, USA) containing 4 types of transcription factors (Klf4, Oct3/4, Sox2, and c-Myc). The used sendai virus was not inserted in the host genome and instead it disappeared after a few sub-cultures, suggesting that more stable iPSCs could be obtained. The dose of sendai virus was determined to be MOI (multiplicity of infection) 3. The cells were infected with sendai virus for overnight, and then the culture medium was replaced with DEM supplemented with 10% FBS, followed by further culture for 6 days for the stabilization of the cells. Then, the cells were transferred to SNL feeder cells (Cell Biolabs, USA) which were the mouse embryonic fibroblasts (MEF) treated with mitomycin C, which were mixed with ESC/iPSC medium (KnockOut™, USA) supplemented with 4 ng/ml of bFGF. The medium was replaced with a fresh one every day during the culture. 30 days after the sendai virus infection, iPSC-like cell colonies were selected and separated. The separated iPSCs proceeded to nucleotide sequence analysis to confirm whether or not the CMT causing gene mutation was retained.

As a result, as shown in Table 1, FIG. 3, and FIG. 4, the CMT patient originated iPSCs (CMT 2F-iPSC) displayed the mutation of 404C>T or 545C>T in HSP27 gene, and the HSP27 protein synthesized therefrom displayed the formation of mutant form wherein the mutation of S135F or P182L was found (Table 1 and FIG. 3). Also, the iPSCs differentiated from the fibroblasts separated from normal group and CMT patients were confirmed to have either the morphology of flat pebble or the same morphology as that of general human pluripotent stem cell (FIG. 4).

<2-2> Expression of CMT 2F-iPSC Endogenous Pluripotent Gene

To investigate whether or not CMT 2F-iPSCs showed pluripotency, the expressions of endogenous genes KLF4, OCT4, SOX2, and c-Myc were confirmed.

Particularly, the CMT 2F-iPSCs prepared in Example 2 or the normal control WA09_hESCs were cultured via 10 cellular passages, followed by suspension in TRIzol (Gibco, USA). Total RNA was extracted from the CMT 2F-iPSCs or WA09_hESC according to the manufacturer's protocol. Then, 1 μg of the extracted RNA and AMV reverse transcriptase (Promega, USA) were mixed with oligo-dT and the forward primer and the reverse primer listed in Table 2, followed by synthesis of cDNA of each KLF4, OCT4, SOX2, and c-Myc gene. The synthesized each cDNA was amplified and the expression of each gene was measured by electrophoresis at the mRNA level.

TABLE 2
Primer sequences for the confirmation of the
pluripotent marker gene expression

| Target | Primer | | | |
|---|---|---|---|---|
| gene | | | | SEQ ID |
| — | Name | Sequence | Direction | NO |
| KLF | KLF | CTG CGG CAA AAC CTA | Forward | SEQ ID |
| | CDR_F | CAC AAA | | NO: 1 |
| | KLF | GCG AAT TTC CAT CCA | Reverse | SEQ ID |
| | CDR_R | CAG CC | | NO: 2 |
| | KLF4 | CAT GGT CAA GTT CCC | Forward | SEQ ID |
| | UTR_F | AAC TGA | | NO: 3 |
| | KLF4 | CAC AGA CCC CAT CTG | Reverse | SEQ ID |
| | UTR_R | TTC TTT G | | NO: 4 |
| Oct3/4 | Oct3/4 | CAG TGC CCG AAA CCC | Forward | SEQ ID |
| | CDR_F | ACA C | | NO: 5 |
| | Oct3/4 | GGA GAC CCA GCA GCC | Reverse | SEQ ID |
| | CDR_R | TCA AA | | NO: 6 |
| | Oct3/4 | GAA AAC CTG GAG TTT | Forward | SEQ ID |
| | UTR_F | GTG CCA | | NO: 7 |
| | Oct3/4 | TCA CCT TCC CTC CAA | Reverse | SEQ ID |
| | UTR_R | CCA GTT | | NO: 8 |
| Sox2 | Sox2 | TAC CTC TTC CTC CCA | Forward | SEQ ID |
| | CDR_F | CTC C | | NO: 9 |
| | Sox2 | GGT AGT GCT GGG ACA | Reverse | SEQ ID |
| | CDR_R | TGT GA | | NO: 10 |
| | Sox2 | CCC GGT ACG CTC AAA | Forward | SEQ ID |
| | UTR_F | AAG AA | | NO: 11 |
| | Sox2 | GGT TTT TGC GTG AGT | Reverse | SEQ ID |
| | UTR_R | GTG GAT | | NO: 12 |
| c-Myc | c-Myc | CGT CCT CGG ATT CTC | Forward | SEQ ID |
| | CDR_F | TGC TC | | NO: 13 |
| | c-Myc | GCT GGT GCA TTT TCG | Reverse | SEQ ID |
| | CDR_R | GTT GT | | NO: 14 |
| | c-Myc | GCG TCC TGG GAA GGG | Forward | SEQ ID |
| | UTR_F | AGA TCC GGA GC | | NO: 15 |
| | c-Myc | TTG AGG GGC ATC GTC | Reverse | SEQ ID |
| | UTR_R | GCG GGA GGC TG | | NO: 16 |

As a result, as shown in FIG. 5, the expressions of endogenous genes KLF4, OCT4, SOX2, and c-Myc were confirmed (FIG. 5).

<2-3> Expression of CMT 2F-iPSC Pluripotency Marker Protein

To confirm the stem cell marker in the CMT originated iPSCs, the expressions of stemness marker proteins SSEA4 and NANOG were additionally investigated.

Particularly, the CMT 2F-iPSCs prepared in Example 2 or the normal control WA09_hESCs were mixed with SNL cells in a gelatin coated chamber slide (Lab-Tek II), followed by culture. One week later, the cultured cells were fixed with 4% paraformaldehyde, followed by immunostaining using 10% normal goat serum (NGS; Gibco, USA) and 0.2% triton X-100. The primary antibodies used herein were anti-SSEA4 antibody (mouse IgG3, 1:100; MC-813-70, DSHB, USA) and anti-NANOG antibody (mouse IgG1, 1:500; NNG-811, Abcam, USA). Cy3-conjugated goat derived anti-mouse IgG secondary antibody and DAPI counterstain were used for visualization.

As a result, as shown in FIG. 6, it was confirmed that both NANOG protein that used to be expressed in nucleus and SSEA4 that used to be expressed in plasma membrane were expressed in the CMT 2F-iPSCs significantly (FIG. 6).

<2-4> Differentiation of EB and Tissues from CMT 2F-iPSCs

To confirm the pluripotency of CMT 2F-iPSCs in vitro, the differentiation of EB was induced from CMT 2F-iPSCs, and then the differentiations of ectoderm, mesoderm, and endoderm originated tissues were also induced from the differentiated EB.

Particularly, the CMT 2F-iPSCs prepared in Example 2 or the normal control WA09_hESCs were transferred in the uncoated Petri-dish having the bottom floor where cells are not easily attached, followed by culture for 8 days with replacing ESC/iPSC medium (KnockOut™, Gibco, USA) every two days. The suspended cells were obtained as embryoid body (EB).

The obtained EB was transferred into the gelatin coated chamber slide (Lab-Tek), followed by culture for 8 days in 10% FBS/DMEM to induce the differentiation into ectoderm, mesoderm, and endoderm originated tissues.

The differentiated cells proceeded to immunostaining performed by the same manner as described in Example <2-3>. The primary antibodies used herein were anti-alpha fetoprotein Ab (anti-AFP Ab, mouse IgG2b, 1:100; 2A9, Abcam, USA), anti-alpha smooth muscle actin Ab (mouse IgG2a, 1:100; 1A4, Abcam, USA), and anti-Nestin Ab (mouse IgG1, 1:1000; 10C2, Abcam, USA), and the secondary antibody used for the reaction was FITC-conjugated goat derived anti-mouse IgG antibody. Upon completion of the reaction, the cells were mounted with a solution containing DAPI counterstain, followed by analysis under confocal microscope.

As a result, as shown in FIG. 7, EB differentiated from CMT patient originated iPSCs was obtained. It was confirmed that α-fetoprotein (AFP) (endoderm), smooth muscle actin (SMA) (mesoderm), and Nestin (Ectoderm) were successfully expressed in the EB (FIG. 7).

<2-5> Confirmation of Differentiation Potency of CMT 2F-iPSCs In vivo

To confirm the differentiation potency of CMT 2F-iPSCs in vivo, the teratoma formation of CMT 2F-iPSCs was investigated in the mouse with immune injury.

Particularly, the CMT 2F-iPSCs (S135F and P182L) induced by the same manner as described in Example 2 or the normal control WA09_hESCs were detached as small cell clumps. 1.0×10⁶ cells were counted and mixed with matrigel at the ratio of 1:1 (v/v). The mixed matrigel-cell mixture was injected in a 5 week old female immunodeficient mouse (NOD/SCID mouse) hypodermically under the back. The xenografted mouse was raised for 8 weeks. The mouse was sacrificed and the generated teratoma was explanted and fixed in 10% natural buffered formaldehyde (10% NBF) for overnight. Then, paraffin blocks were prepared. The paraffin blocks were cut into 0.4 μm thick sections, followed by Hematoxylin and Eosin (H&E) staining for further observation.

As a result, as shown in FIG. 8, the CMT 2F-iPSCs injected in the mouse formed teratoma peculiarly and were also differentiated into ectoderm, mesoderm, and endoderm originated tissues, suggesting that the CMT patient originated iPSCs had in vivo pluripotency (FIG. 8).

### EXAMPLE 3

### Inducement of the Differentiation of CMT Patient Originated Motor Neurons and the Differentiation Efficiency Thereof

<3-1> Differentiation of Motor Neurons from CMT 2F-iPSCs

To use CMT 2F-iPSCs as the peripheral neuropathy model, the differentiation of motor neurons from CMT 2F-iPSCs was induced by the same manner as described in FIG. 2 (Amoroso M W, et al, J Neurosci 2013; 33: 574-586).

Particularly, the CMT 2F-iPSCs (S135F and P182L) induced by the same manner as described in Example 2 or the normal control WA09_hESCs were separated as small clumps, followed by suspension culture in ESC/iPSCs medium (basal medium) supplemented with 10 μM Y27632 (Rho-associated kinase inhibitor, Tocris Bioscience, Great Britain), 20 ng/ml bFGF (Invitrogen, USA), 10 μM SB435142 (Stemgent, USA), 0.2 μM LDN193189 (Stemgent, USA), and penicillin/streptomycin for 2 days in order to induce the formation of embryoid body.

3 days after the culture began, the basal medium was replaced with Neural stem cell medium (Stemline; Sigma, USA), to which 2 μg/ml of heparin (Sigma, USA) and N2 supplement (Gibco, USA) were added in order to induce neuralization. 1 μM retinoic acid (Sigma, USA), 0.4 μg/ml of ascorbic acid (Sigma, USA), and 10 ng/ml of BDNF (R&D, USA) were added thereto, followed by caudalization to obtain neurosphere.

Then, 7 days after the culture began, 10 μM SB435142 and 0.2 μM LDN193189 were stopped to be added. Instead, purmorphamine (Stemgent, USA), the sonic hedgehog (shh) agonist, was added thereto, followed by culture for ventralization.

17 days after the culture began, the basal medium was replaced with neurobasal medium (Invitrogen, USA). While the addition of all the said constituents continued, 10 ng/ml of IGF-1, 10 ng/ml of GDNF, 10 ng/ml of CNTF (R&D, USA), and B27 supplement (Gibco, USA) were additionally added thereto in order to differentiate the neurosphere into motor neurons. The cells were maintained as suspended in the culture fluid during the culture. 20 or 30 days after the culture began, the cultured cells were treated with accutase (PAA Laboratories) that made the cells scattered in poly-L-lysine/laminin coated culture vessel or slide chamber (Nalgene Nunc, USA). As a result, the motor neurons (CMT-2F-MN or WA09_MN) differentiated from CMT 2F iPSCs or WA09 hESCs were obtained.

<3-2> Expression of CMT 2F-MN Marker Protein

To confirm the differentiation efficiency of motor neurons differentiated from CMT 2F-iPSCs, the expression of motor neuron marker protein and the formation of neuromuscular junction were investigated.

Particularly, the CMT 2F-MN or WA09_MN obtained in Example <3-1> proceeded to immunostaining by the same manner as described in Example <2-3> in order to confirm the expression of motor neuron marker protein. The primary antibodies used herein were anti-HB9 antibody (mouse IgG1, 1:100; 81.5C10, DSHB, USA), anti-Islet-1/2 antibody (mouse IgG2b, 1:50; 39.4DS, DSHB, USA), anti-SM132 antibody (anti-H-non-phosphorylated neurofilament, mouse IgG1, 1:500; Covance, USA), anti-neuron specific beta III tubulin (Tuj1) antibody (rabbit IgG, 1:1000; Abcam, USA), anti-microtubule-associated protein 2 (anti-MAP2) antibody (rabbit IgG, 1:200; Millipore, USA), anti-synapsin antibody (rabbit IgG, 1:100; Abcam, USA), and anti-choline acetyltransferase (anti-ChAT) antibody (rabbit IgG, 1:1000; Abcam, USA). The secondary antibodies used herein were FITC-conjugated goose anti-mouse IgG, Cy3-conjugated goat anti-rabbit IgG, and Cy3-conjugated goat anti-mouse IgG antibody. DAPI counterstain was used for visualization. To evaluate the degree of the development of motor neurons, the percentage of SM132/DAPI or MAP2/DAPI was calculated. The length of axon was also measured for the comparison.

As a result, as shown in FIG. 9, the motor neurons differentiated from the normal control and CMT 2F-iPSCs were confirmed to express significantly the motor neuron marker proteins HB9, ISL1, SM132, Tuj1, MAP2 Synapsin, and ChAT (FIG. 9a). The differentiated CMT 2F-MN was not so much different from the normal control, suggesting that there was no developmental defect in the course of differentiation (FIG. 9b and FIG. 9c).

<3-2> Formation of CMT 2F-MN Neuromuscular Junction

To confirm the differentiation efficiency of motor neurons differentiated from CMT 2F-iPSCs, the formation of neuromuscular junction was investigated.

Particularly, C2C12 mouse myoblasts (CRL-1772, ATCC) were cultured in DMEM supplemented with 10% FBS, 1 mM glutamine, and penicillin/streptomycin. When the cells were grown to 70% confluency, 1% insulin-transferrin-selenium (ITS) supplement (Sigma, USA) was added to the culture medium to induce the differentiation of myotubes. After culturing the cells for 2 days, 10 μM cytosine arabinoside was added thereto in order to eliminate dividing cells, followed by further culture for 2˜4 days. Then, the differentiated myotubes were obtained by using trypsin, which were inoculated in a matrigel-coated 8-well slide chamber at the low density of 1.0×10⁴ cells/well. One or two days later, the CMT 2F-MN or WA09_MN obtained in Example <3-1> was added to the inoculated myotubes, followed by co-culture at the ratio of 10:1. Then, MN differentiation medium was added thereto. One week later, the co-cultured motor neurons and myotubes were stained with Alexa 488-conjugated α-bungarotoxin (α-BTX; Invitrogen, USA) to observe the newly formed neuromuscular junction.

As a result, as shown in FIG. 10, it was confirmed that the CMT-2F-MN co-cultured with myotubes normally formed neuromuscular junction (FIG. 10).

### EXAMPLE 4

### Recovery of Axonal Transport in CMT Originated Motor Neurons by Histon Deacetylase 6 (HDAC6) Inhibitor

<4-1> Acetylation of CMT 2F-MN α-tubulin

To use the neurons differentiated from CMT patient originated iPSCs as the CMT drug efficiency test model, the recovery of axonal transport according to the treatment of tubastatin A, the histon deacetylase 6 (HDAC6) inhibitor, was investigated. CM2 subtype has heterogeneity in CMT causing gene, but nevertheless it causes malfunction in axonal transport system in many patients (Gentil B J and Cooper L, Brain Res Bull 2012; 88: 444-453). Therefore, the axonal transport efficiency of CMT 2F-MN was investigated by measuring the level of α-tubulin acetylation which was reported previously to be associated with the interaction between the vehicle and the motor protein (Westermann S and Weber K. Nat Rev Mol Cell Biol 2003; 4: 938-947).

Particularly, the CMT-2F-MN or WA09_MN differentiated by the same manner as described in Example <3-1> was treated with 5 μM tubastatin A, followed by culture for 12 hours. Then, the cells were immunostained with α-tubulin and acetylated α-tubulin by the same manner as described in Example <2-3>. The primary antibodies used herein were anti-α-tubulin antibody (rabbit IgG, 1:500; Abcam, USA) and anti-acetylated α-tubulin antibody (mouse IgG, 1:200; Abcam, USA). The secondary antibodies used herein were Alexa 488-conjugated goat anti-rabbit IgG and Cy3-conjugated goat anti-mouse IgG antibody.

The CMT-2F-MN or WA09_MN treated with 5 μM tubastatin A was suspended in RIPA lysis buffer (pH 8.0) containing 150 mM NaCl, 1.0% NP-40, 0.5% sodium deoxycholate, 0.1% sodium dodecylsulfate, and 50 mM Tris. Then, the supernatant containing cellular proteins was obtained, and the proteins were separated on 12% SDS-PAGE gel. The proteins were transferred onto PVDF membrane. The membrane proceeded to immunoblotting using anti-acetylated α-tubulin antibody (mouse IgG2b, 1:1000; 6-11B-1, Abcam) and anti-α-tubulin antibody (rabbit, mouse IgG1, 1:1000; DM1A, Sigma, USA). The band density was analyzed by using UN-SCAN-IT gel software in order to (Silk Scientific, USA) measure the level of α-tubulin acetylation. As for the negative control, the CMT-2F-MN or WA09_MN not-treated with 5 μM tubastatin A was immunostained by the same manner as described above, followed by immunoblotting.

As a result, as shown in FIG. 11, when CMT 2F-MN was not treated with tubastatin A, the level of α-tubulin acetylation was reduced, compared with the normal control WA09_MN. In the meantime, when CMT-2F-MN was treated with 5 μM tubastatin A, the level of α-tubulin acetylation was increased/recovered back to that of the normal control group (FIGS. 11a, 11b, and 11c).

<4-2> Moving Mitochondria of CMT 2F-MN α-tubulin

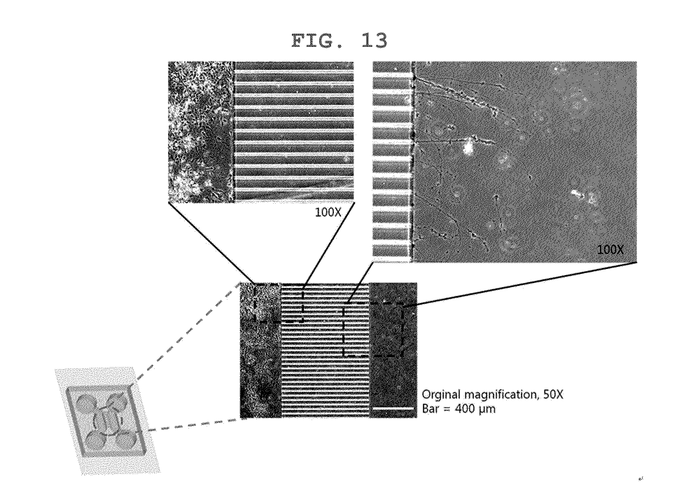

To use the neurons differentiated from CMT patient originated iPSCs as the CMT drug efficiency test model, moving mitochondria was investigated over the treatment of tubastatin A, the histon deacetylase 6 (HDAC6) inhibitor, through microfluidic culture, as shown in FIG. 13. And the axonal transport efficiency of motor neurons (CMT 2F-MN) was confirmed (FIG. 13).

Particularly, the CMT-2F-MN or WA09_MN obtained in Example <3-1> was separated as single cells by using accutase, which were then inoculated in microchannel plates (provided by Dr. Mok, Seoul National University, Korea; Park J W et al., Nat Protoc 2006; 1: 2128-2136) at the density of 1.0×10⁵ cells/plate, followed by culture in neurobasal/B27 for 10 days. After axons were fully grown through micrometer-sized grooves and stretched to the opposite compartment, the processed motor neurons were transfected with mito-dsRED2 by using lipofectamine 2000 (Invitrogen, USA). Within 2 days from the transfection, 5˜10 μM tubastatin A was treated to the medium, followed by culture for 6 hours. Imaging of mitochondria was performed by using fluorescent microscope at the speed of 121 snaps/2 min. Moving velocity of the motor neuron was measured by using ImageJ and Kymograph.

As a result, as shown in FIG. 12, Table 3 and Table 4, axonal mitochondria of motor neuron was observed in mito-RED2 transfected CMT-2F-MN or

WA09_MN. When tubastatin A was not treated, moving velocity of mitochondria was significantly reduced in CMT 2F-MN axons having the mutation of S135F. In CMT 2F-MN having the mutation of P182L, the percentage of moving mitochondria was reduced (FIGS. 12a, 12b, and 12c). On the other hand, when tubastatin A was treated, moving velocity and transport frequency of mitochondria were significantly increased in both CMT 2F-MNs respectively having the mutation of S135F and the mutation of P182L, which were recovered almost to the level of normal control (FIGS. 12b and 12c).

TABLE 3
Moving velocity of mitochondria over the treatment of tubastatin A

Moving velocity (μm/sec)

| | Tubastatin A | 5 μM tubastatin |
|---|---|---|
| ID No. | non-treated | A treated |
| WA09_hESC- | 0.2389 ± 0.013310 | 0.2446 ± 0.038590 |
| MN | | |
| HSP27 | 0.1427 ± 0.009589 | 0.2498 ± 0.023570 |
| S135F-MN | | |
| HSP27 | 0.2244 ± 0.009310 | 0.2599 ± 0.051860 |
| P182L-MN | | |

TABLE 4
Migration of mitochondria over the treatment of tubastatin A

Migration (%)a

| | Tubastatin A | 10 μM tubastatin |
|---|---|---|
| ID NO. | non-treated | A treated |
| WA09_hESC- | 31.39 ± 3.741 | 39.31 ± 3.831 |
| MN | | |
| HSP27 | 22.14 ± 6.410 | — |
| S135F-MN | | |
| HSP27 | 14.64 ± 2.136 | 44.61 ± 10.450 |
| P182L-MN | | |

aMigration is presented as percentage (%) of the number of moving mitochondria by the total number of mitochondria.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended Claims.

What is claimed is:

1. A method for preparation of motor neurons from somatic cells originated from a Charcot-Marie-Tooth disease (CMT) patient, wherein the method comprises the following steps:
1) obtaining human somatic cells from the Charcot-Marie-Tooth disease (CMT) patient;
2) transfecting the human somatic cells originated from the CMT patient of step 1) with a vector comprising OCT4, SOX2, KLF4, and c-MYC transgenes, followed by culturing to induce induced pluripotent stem cells (iPSC); and
3) culturing the induced pluripotent stem cells prepared in step 2) in the presence of retinoic acid and sonic hedgehog to induce motor neurons.

2. A method for preparation of motor neurons from somatic cells originated from a Charcot-Marie-Tooth disease (CMT) patient, wherein the method comprises the following steps:
1) obtaining human somatic cells from the Charcot-Marie-Tooth disease (CMT) patient;
2) transfecting the human somatic cells originated from the CMT patient of step 1) with a vector comprising OCT4, SOX2, KLF4, and c-MYC transgenes, followed by culturing to induce induced pluripotent stem cells (iPSC);
3) culturing the induced pluripotent stem cells prepared in step 2) in the presence of retinoic acid and sonic hedgehog to induce motor neurons; and
4) extending the culture of the motor neurons prepared in step 3) in the presence of neurotrophin.

3. The method for the preparation of motor neurons according to claim 2, wherein the neurotrophin of step 4) is selected from the group consisting of nerve growth factor (NGF), brain-derived neurotrophic factor (BDNF), neurotrophin-3 (NT-3), and glial cell-derived neurotrophic factor (GDNF).

4. The method for the preparation of motor neurons according to claim 1, wherein the Charcot-Marie-Tooth disease is CMT type I, CMT type II, CMT type IV, or CMTX.

5. The method for the preparation of motor neurons according to claim 1, wherein the human somatic cells of step 1) are characteristically fibroblasts.

6. The method for the preparation of motor neurons according to claim 1, wherein the vector of step 2) is a sendai virus, a retrovirus, or a lentivirus.

7. The method for the preparation of motor neurons according to claim 4, wherein the CMT type II has the mutation of the 135^{th} amino acid or the 182^{nd} amino acid in heat-shock protein (HSP) 27.

8. The method for the preparation of motor neurons according to claim 1, wherein step 3) is composed of the following substeps:
(3-1) culturing the induced pluripotent stem cells to obtain embryoid body (EB) and then differentiating the obtained EB into neurosphere; and
(3-2) differentiating the neurosphere into motor neurons.

9. A screening method for a composition for the prevention and treatment of Charcot-Marie-Tooth disease comprising the following steps:
1) treating the motor neurons prepared by the method of claim 1 with CMT treatment material candidates in vitro;
2) measuring the CMT index in the cells treated with the treatment material candidates in step 1); and
3) selecting a candidate that displays an increase or decrease of the CMT index obtained in step 2) by comparing with the control.

10. The screening method for a composition for the prevention and treatment of Charcot-Marie-Tooth disease according to claim 9, wherein the Charcot-Marie-Tooth disease is CMT type I, CMT type II, CMT type IV, or CMTX.

11. The screening method for a composition for the prevention and treatment of Charcot-Marie-Tooth disease according to claim 10, wherein the CMT 2F has the mutation of the 135^{th} amino acid or the 182^{nd} amino acid in heat-shock protein (HSP) 27.

12. The screening method for a composition for the prevention and treatment of Charcot-Marie-Tooth disease according to claim 9, wherein the CMT index is either acetylated α-tubulin, an axonal transport index, or moving mitochondria.

13. The screening method for a composition for the prevention and treatment of Charcot-Marie-Tooth disease according to claim 9, wherein step 3) is characterized by selection of those candidates that can increase CMT index such as acetylated α-tubulin, the axonal transport index, and moving mitochondria.

14. The screening method for a composition for the prevention and treatment of Charcot-Marie-Tooth disease according to claim 9, wherein the measurement of CMT index is performed by one of the methods selected from the group consisting of RT-PCR, ELISA, immunohistochemistry (IHC), Western blotting, FACS, and whole cell patch clamp.

15. A screening method for a CMT patient specific treating material comprising the following steps:
1) treating the motor neurons prepared by the method of claim 1 in vitro with CMT treating drugs;
2) measuring CMT index level in the cells treated with CMT treating drugs of step 1); and
3) selecting those CMT treating drugs that increased or reduced CMT index level in step 2) by comparing the level of the control.

16. The method for the preparation of motor neurons according to claim 2, wherein the Charcot-Marie-Tooth disease is CMT type I, CMT type II, CMT type IV, or CMTX.

17. The method for the preparation of motor neurons according to claim 2, wherein the human somatic cells of step 1) are characteristically fibroblasts.

18. The method for the preparation of motor neurons according to claim 2, wherein the vector of step 2) is a sendai virus, a retrovirus, or a lentivirus.

19. The method for the preparation of motor neurons according to claim 2, wherein step 3) is composed of the following substeps:
(3-1) culturing the induced pluripotent stem cells to obtain embryoid body (EB) and then differentiating the obtained EB into neurosphere; and
(3-2) differentiating the neurosphere into motor neurons.

20. A screening method for a composition for the prevention and treatment of Charcot-Marie-Tooth disease comprising the following steps:
1) treating the motor neurons prepared by the method of claim 2 with CMT treatment material candidates in vitro;
2) measuring the CMT index in the cells treated with the treatment material candidates in step 1); and
3) selecting a candidate that displays an increase or decrease of the CMT index obtained in step 2) by comparing with the control.

21. A screening method for a CMT patient specific treating material comprising the following steps:
1) treating the motor neurons prepared by the method of claim 2 in vitro with CMT treating drugs;
2) measuring CMT index level in the cells treated with CMT treating drugs of step 1); and
3) selecting those CMT treating drugs that increased or reduced CMT index level in step 2) by comparing the level of the control.


SEQUENCE LISTING (16 sequences)

SEQ ID NO: 1 - DNA, 21 bp, Artificial Sequence
  Primer
ctgcggcaaaacctacacaaa

SEQ ID NO: 2 - DNA, 21 bp, Artificial Sequence
  Primer
ctgcggcaaaacctacacaaa

SEQ ID NO: 3 - DNA, 21 bp, Artificial Sequence
  Primer
ctgcggcaaaacctacacaaa

SEQ ID NO: 4 - DNA, 21 bp, Artificial Sequence
  Primer
ctgcggcaaaacctacacaaa

SEQ ID NO: 5 - DNA, 21 bp, Artificial Sequence
  Primer
ctgcggcaaaacctacacaaa

SEQ ID NO: 6 - DNA, 21 bp, Artificial Sequence
  Primer
ctgcggcaaaacctacacaaa

SEQ ID NO: 7 - DNA, 21 bp, Artificial Sequence
  Primer
ctgcggcaaaacctacacaaa

SEQ ID NO: 8 - DNA, 21 bp, Artificial Sequence
  Primer
ctgcggcaaaacctacacaaa

SEQ ID NO: 9 - DNA, 21 bp, Artificial Sequence
  Primer
ctgcggcaaaacctacacaaa

SEQ ID NO: 10 - DNA, 21 bp, Artificial Sequence
  Primer
ctgcggcaaaacctacacaaa

SEQ ID NO: 11 - DNA, 21 bp, Artificial Sequence
  Primer
ctgcggcaaaacctacacaaa

SEQ ID NO: 12 - DNA, 21 bp, Artificial Sequence
  Primer
ctgcggcaaaacctacacaaa

SEQ ID NO: 13 - DNA, 21 bp, Artificial Sequence
  Primer
ctgcggcaaaacctacacaaa

SEQ ID NO: 14 - DNA, 21 bp, Artificial Sequence
  Primer
ctgcggcaaaacctacacaaa

SEQ ID NO: 15 - DNA, 21 bp, Artificial Sequence
  Primer
ctgcggcaaaacctacacaaa

SEQ ID NO: 16 - DNA, 21 bp, Artificial Sequence
  Primer
ctgcggcaaaacctacacaaa